(19) United States
(12) Patent Application Publication
(10) Pub. No.: US 2025/0213840 A1
(43) Pub. Date: Jul. 3, 2025

(54) Removable, Adjustable Grip Clamp for a Pen Style Tattoo Machine and Methods of Making and Using Same

(71) Applicant: Tyler N. Tokach, Kingston, PA (US)

(72) Inventor: Tyler N. Tokach, Kingston, PA (US)

(21) Appl. No.: 18/957,770

(22) Filed: Nov. 24, 2024

Related U.S. Application Data
(60) Provisional application No. 63/617,344, filed on Jan. 3, 2024.

(51) Int. Cl. A61M 37/00

(52) U.S. Cl. CPC A61M 37/0076

(57) ABSTRACT

Systems, methods, and other embodiments for a pen style tattoo machine having a removable, adjustable grip clamp assembly, including a tattoo machine having a tattoo needle, a grip assembly having an back stem located at one end of the grip assembly and a tattoo needle cartridge located at another end of the grip assembly, wherein the grip assembly is configured to allow the tattoo needle to extend out of the tattoo needle cartridge, and a removable, adjustable grip clamp assembly removably attached to the pen style tattoo machine and the back stem on the grip assembly.

### CROSS-REFERENCE TO RELATED APPLICATION

This application claims the benefit of U.S. Patent Application 63/617,344, filed on Jan. 3, 2024, the disclosure of which is hereby incorporated by reference in its entirety to provide continuity of disclosure to the extent such disclosure is not inconsistent with the disclosure herein.

### FIELD OF THE INVENTION

The present invention relates to a removable, adjustable grip clamp for pen style tattoo machines. In particular, the removable, adjustable grip clamp for a pen style tattoo machine allows the tattoo artist to be able to attach a disposable grip or a tube with a back stem to a pen style tattoo machine. In this manner, the tattoo artist will be able to choose to use either a disposable grip with a tube with a back stem or the reusable grip on the pen style tattoo machine for a tattoo application session.

### BACKGROUND OF THE INVENTION

It is known that pen style tattoo machines can be a cross-contamination hazard for several reasons. First, the inner workings of the pen style tattoo machine often cannot be accessed to clean the push rod and machine housing of the pen style tattoo machine. Second, disposable pen style tattoo machine grips are often not an option to be able to use or buy. For example, when a manufacturer makes a disposable grip for a pen style tattoo machine, those grips are made only for the manufacturer's specific pen style tattoo machine and not for their competitor's pen style tattoo machines. This problem is further exacerbated by the fact that these disposable grips often sell out quickly due to the high demand, which results in the tattoo artist having to use the metallic, reusable grips that are originally equipped with the pen style tattoo machine.

The prior art issues discussed above create several problems for tattoo artists. First, some tattoo artists do not properly clean and sanitize reusable grips. This often results in cross-contamination which can create a serious illness or infection in the client and the tattoo artist. Second, the use of reusable grips is inconvenient. Many tattoo artists prefer disposable grips because they save time during the set-up and tear down of the tattoo station. Also, many artists prefer disposable grips for the simple fact that they are fully clean every time and do not require extensive cleaning like reusable grips.

Therefore, it would be desirable to provide a removable, adjustable grip clamp for a pen style tattoo machine that allows the tattoo artist to be able to remove a tattoo machine grip quickly and easily from the pen style tattoo machine and quickly and easily replace the tattoo machine grip with a disposable grip or a tube with back stem.

This invention's purpose is to fulfill these and other needs in the pen style tattoo machine art in a manner more apparent to the skilled artisan once given the following disclosure.

The preferred removable, adjustable grip clamp for a pen style tattoo machine, according to various embodiments of the present invention, offers the following advantages: ease of use; portability; ease of removing and replacing the tattoo machine grip; ease of removing and replacing the tube with a back stem; disposability; improved cleanliness; reduced likelihood of cross-contamination; reduced cost; ease of attaching the disposable tattoo machine grip to the tattoo machine; ease of attaching the tube with a back stem; and reduced tattoo session time. In many of the preferred embodiments, these advantages are optimized to the extent that it is considerably higher than previously achieved in prior known pen style tattoo machines.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 is a schematic, front view of a pen style tattoo machine, constructed according to the prior art.

FIG. 2 is a schematic, exploded view of the pen style tattoo machine with its reusable grip removed, constructed according to the prior art.

FIG. 3 is another schematic, exploded view of the pen style tattoo machine, constructed according to the prior art.

FIG. 4 is a schematic, front view of a drive assembly of a pen style tattoo machine, constructed according to the prior art.

FIG. 5 is a schematic, exploded view of the drive assembly of a pen style tattoo machine, according to various embodiments of the present invention described herein.

FIG. 6 is a schematic, exploded view of a removable, adjustable grip clamp for a pen style tattoo machine, according to various embodiments of the present invention described herein.

FIG. 7 is a schematic, cross-sectional view of a removable, adjustable grip clamp for a pen style tattoo machine, taken along lines 7-7 in FIG. 6, according to various embodiments of the present invention described herein.

FIG. 8 is a schematic, front view of a removable, adjustable grip clamp for a pen style tattoo machine being attached to the motor of the pen style tattoo machine, according to various embodiments of the present invention described herein.

FIG. 9 is a schematic, front view of a removable, adjustable grip clamp for a pen style tattoo machine being attached to a disposable tattoo machine grip, according to various embodiments of the present invention described herein.

FIG. 10 is a schematic, cross-sectional view of the removable, adjustable grip clamp for a pen style tattoo machine being attached to the disposable tattoo machine grip, taken along lines 10-10 in FIG. 9, according to various embodiments of the present invention described herein.

FIG. 11 is a schematic, exploded view of another embodiment of a removable, adjustable grip clamp for a pen style tattoo machine being attached to a disposable tattoo machine grip, according to various embodiments of the present invention described herein.

FIG. 12a is a schematic, front view of another embodiment of the drive assembly of a pen style tattoo machine, according to various embodiments of the present invention described herein.

FIG. 12b is a schematic, exploded view of another embodiment of the removable, adjustable grip clamp for a pen style tattoo machine, according to various embodiments of the present invention described herein.

FIG. 12c is a schematic, top view of another embodiment of a removable, adjustable grip clamp for a pen style tattoo machine, according to various embodiments of the present invention described herein.

FIG. 12d is a schematic, exploded view of another embodiment of the removable, adjustable grip clamp for a pen style tattoo machine, according to various embodiments of the present invention described herein.

FIG. 12e is a schematic, top view of another embodiment of a removable, adjustable grip clamp for a pen style tattoo machine, taken along lines 12e-12e in FIG. 12d, according to various embodiments of the present invention described herein.

FIG. 13 is a schematic, front view of a coil tattoo machine, according to the prior art.

FIG. 14 is a schematic, front view of a removable, adjustable grip clamp for a coil tattoo machine being attached to a disposable tattoo machine grip, according to various embodiments of the present invention described herein.

FIG. 15 is a schematic, exploded view of the removable, adjustable grip clamp for a coil tattoo machine for use with a disposable tattoo machine grip, as shown in FIG. 14, according to various embodiments of the present invention described herein.

FIG. 16 is a schematic, front view of another embodiment of a removable, adjustable grip clamp for a coil tattoo machine being attached to a disposable tattoo machine grip, according to various embodiments of the present invention described herein.

FIG. 17 is a schematic, exploded view of the removable, adjustable grip clamp for a coil tattoo machine for use with a disposable tattoo machine grip, as shown in FIG. 16, according to various embodiments of the present invention described herein.

### DETAILED DESCRIPTION OF THE INVENTION

The terminology used herein is to describe particular embodiments only and is not intended to be limiting to the invention. As used herein, the term “and/or” includes all combinations of one or more of the associated listed items. As used herein, singular forms “a”, “an”, and “the” are intended to include the plural forms as well as the singular forms unless the context clearly indicates otherwise. It will be further understood that the terms “comprises” and/or “comprising,” when used in this specification, specify the presence of stated features, steps, operations, elements, and/or components but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefits, and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

A new and unique removable, adjustable grip clamp for a pen style tattoo machine and methods for making and using are discussed herein. In the following description, for purposes of explanation, numerous specific details are set forth to provide a thorough understanding of the present invention. It will be evident, however, to one skilled in the art that the present invention may be practiced without these specific details.

The present disclosure is considered an exemplification of the invention and is not intended to limit the invention to the specific embodiments illustrated by the figures or description below.

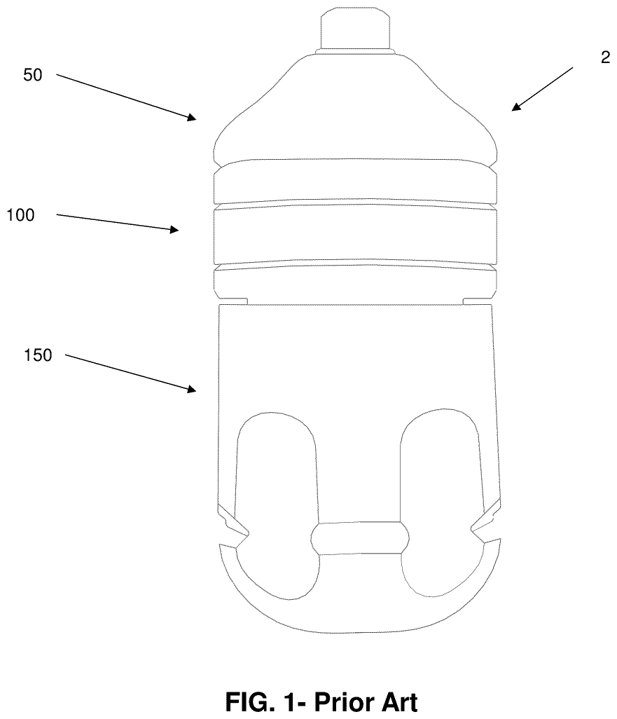

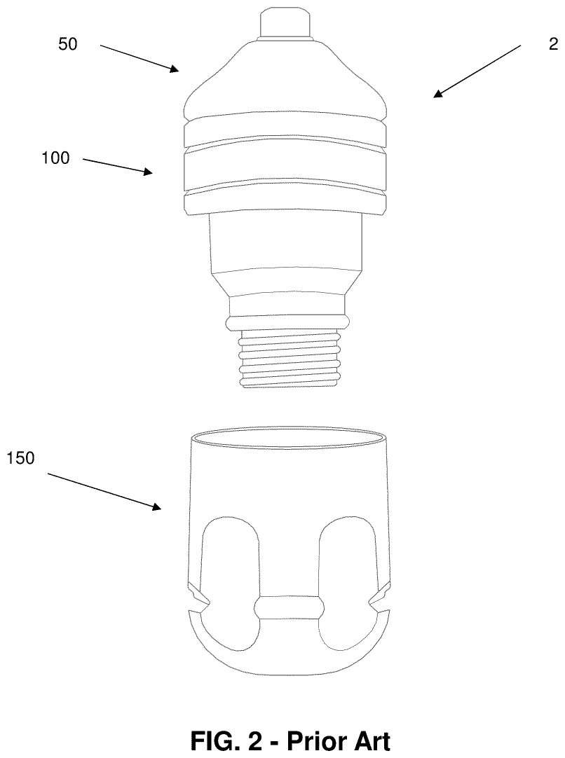

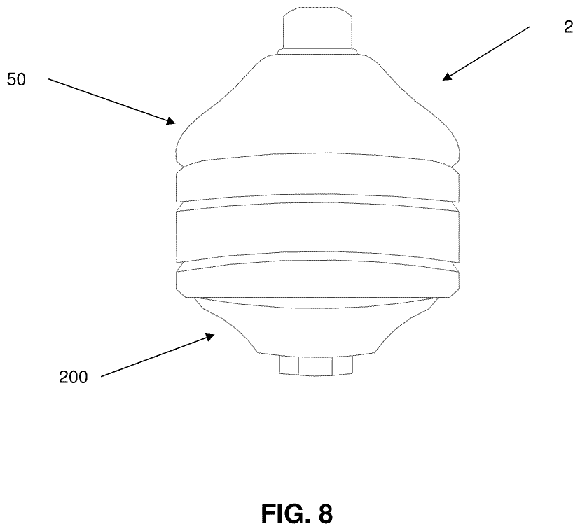

The present invention will now be described by referencing the appended figures representing preferred embodiments. FIGS. 1-17 are illustrations related to a removable, adjustable grip clamp for a pen style tattoo machine and a coil style tattoo machine.

### Pen Style Tattoo Machine

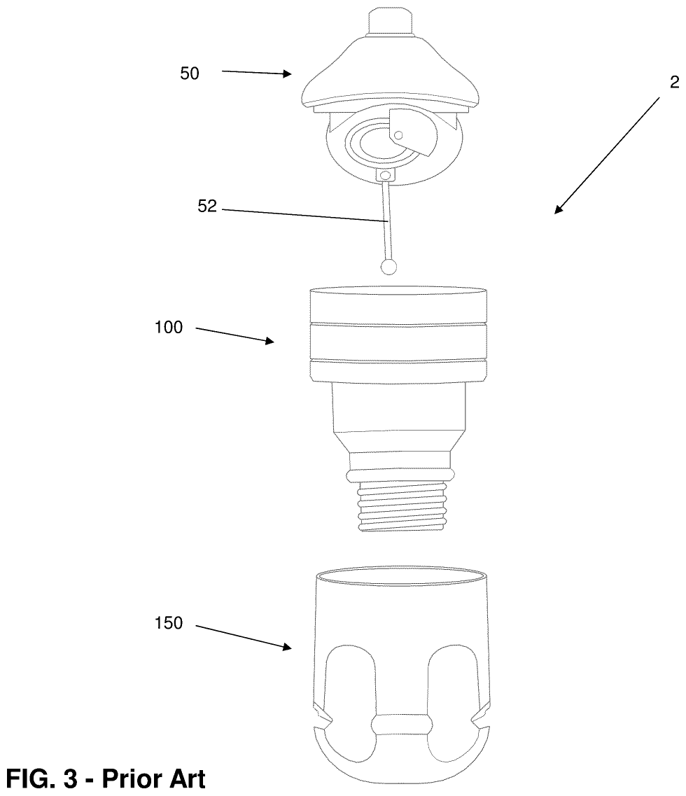

As shown in FIGS. 1-4, there is illustrated a conventional pen style tattoo machine 2. In particular, pen style tattoo machine includes motor assembly 50 having a push rod 52 (FIG. 3), drive assembly 100, and reusable grip 150. A conventional grip assembly (not shown) can be conventionally attached to pen style tattoo machine 2 through drive assembly 100 and reusable grip 150. As is well known in tattoo art, the motor assembly and push rod 52 are utilized to move an ink laden tattoo needle (not shown) located within the grip assembly up and down in order to create the desired tattoo graphic.

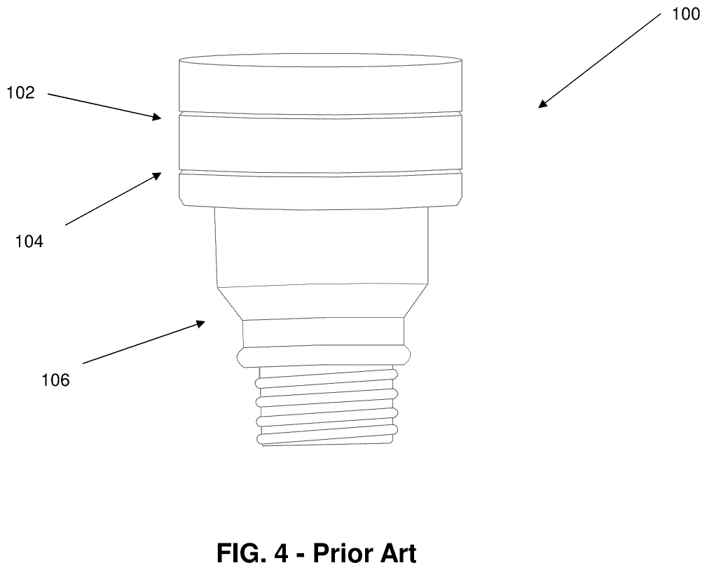

With respect to FIG. 4, drive assembly 100 includes, in part, motor assembly connector 102, driver 104, and housing connector 106. In particular, motor assembly connector 102 is used to removably connect drive assembly 100 to motor assembly 50. Also, housing connector 106 is used to removably connect drive assembly 100 to reusable grip 150.

### Removable, Adjustable Grip Clamp Assembly

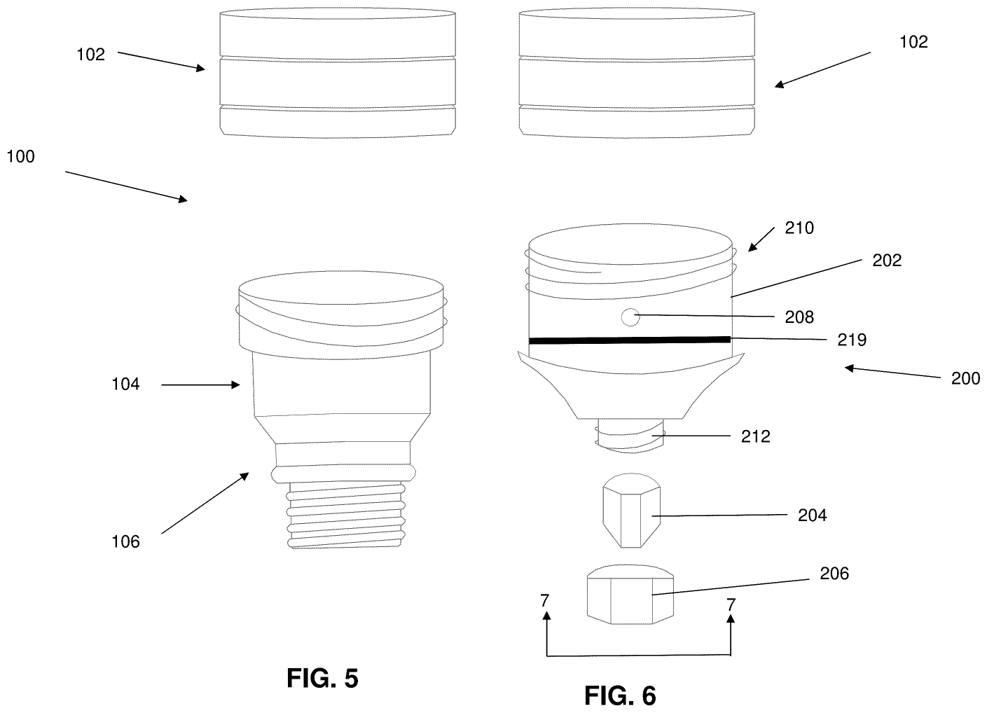

In particular, as shown in FIGS. 5-10, in one embodiment, there is illustrated a removable, adjustable grip clamp assembly 200. In particular, removable, adjustable grip clamp assembly 200 includes, in part, clamp base 202, clamp 204, and fastener 206. Preferably, removable, adjustable grip clamp assembly 200 is constructed of any suitable, durable, high strength, rust resistant, UV resistant, lightweight material.

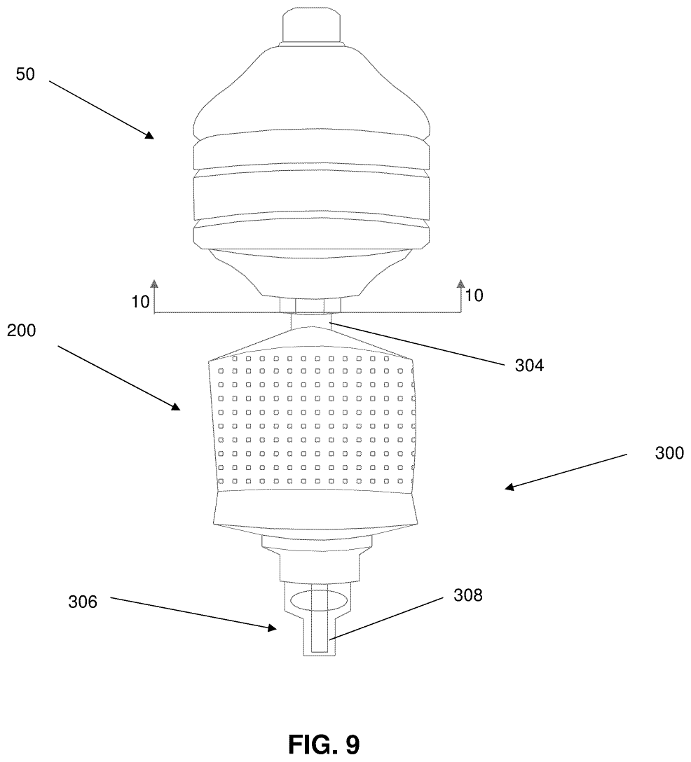

A unique aspect of the present invention is that removable, adjustable grip clamp assembly 200 will allow the pen style tattoo machine 2 to accept and utilize disposable tattoo machine grips that are used normally on traditional style tattoo machines. Not only will this allow the tattoo artists to use the disposable tattoo machine grips 300 (FIG. 9) with pen style tattoo machines 2, but it will also allow for the disposable tattoo machine grip to spin during the tattoo application process in order to change the distance the tattoo machine needle 308 hangs out of the tip of the tattoo needle cartridge 306 (FIG. 9).

With respect to clamp base 202, as shown in FIG. 6, in one embodiment, clamp base 202 includes, in part, button spring 208, threaded area 210, and threaded area 212. As discussed in greater detail later, button spring 208 and threaded area 210 which are located on clamp base 202 in order to interact with motor assembly connector 102 so that removable, adjustable grip clamp assembly 200 can be quickly and easily removed from motor assembly 50 and quickly and securely attached to motor assembly 50. In another embodiment, an O-ring 219 can also be used to securely retain the removable, adjustable grip clamp assembly 200 on the motor assembly 50. Preferably, threaded area 210 and threaded area 212 are formed on clamp base 202 by conventional forming techniques such as grinding, forming, molding or the like.

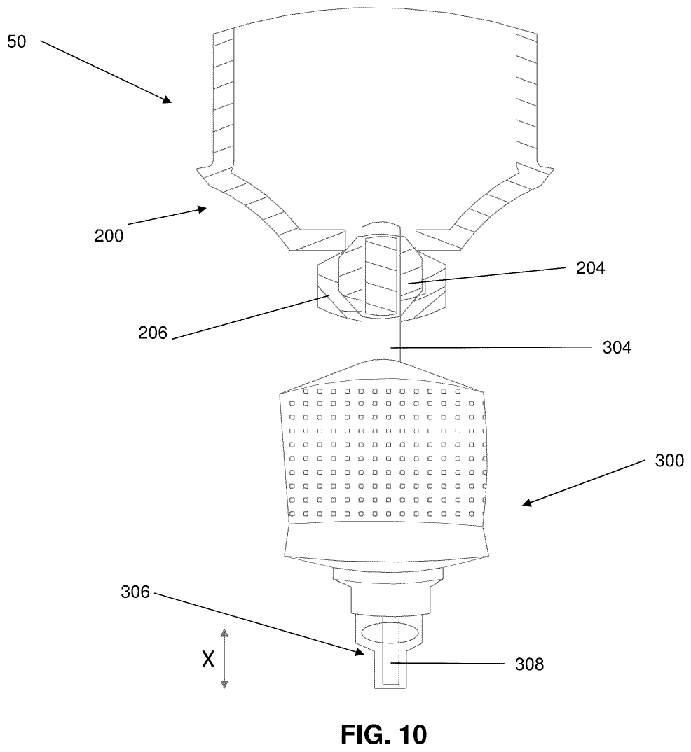

Another unique aspect of the present invention is the use of button spring 208. In particular, button spring 208 can be utilized during the adjustment to change a distance that the tattoo machine needle 308 hangs out of the tip of the tattoo needle cartridge 306 (FIGS. 9 and 10). For example, the clamp base 202 can be twisted in order to adjust the distance that the tattoo machine needle 308 hangs out of the tip of the tattoo needle cartridge 306 (FIG. 9) by making the clamp base 202 and grip assembly 300 move up and down along the direction of arrows X (FIG. 10). In this manner, a ratchet system (not shown) can be used in conjunction with the button spring 208 to allow the clamp base 202 to be rotated a desired amount and the button spring 208 and the ratchet system can be used to retain the clamp base 202 in the desired position. Furthermore, a “clicking” sound produced when the button spring 208 interacts with the ratchet system will provide an audible indication as to how many turns have been made by the clamp base 202 with respect to the motor assembly connector 102 so that the tattoo artist will know how far the clamp base 202 and grip assembly 300 has been moved up or down. For example, two (2) audible “clicks” could be interpreted by the tattoo artist that the clamp base 202 and grip assembly 300 has been moved up or down 1/16th of an inch.

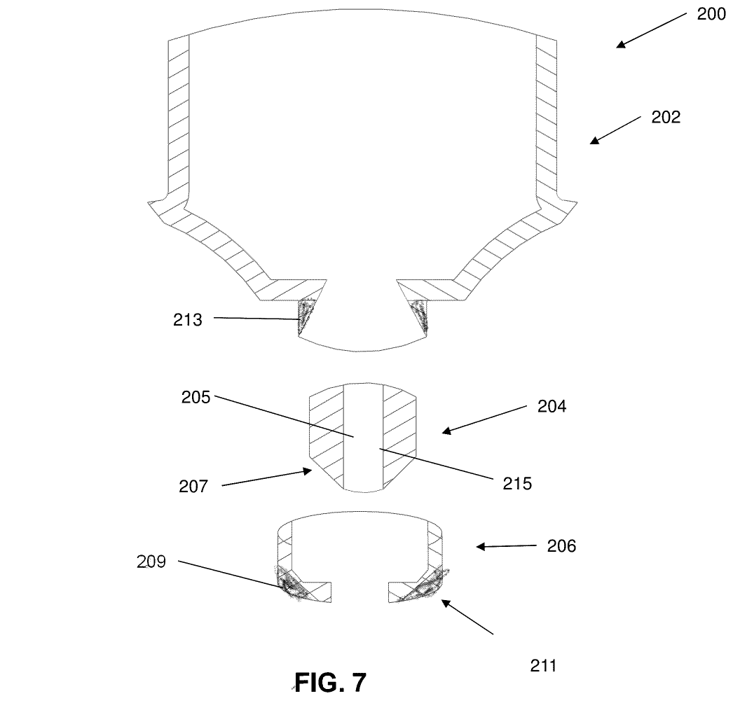

A yet another unique aspect of the present invention is the use of beveled areas 213 (FIG. 7) on clamp base 202. As will be discussed in greater detail later, beveled areas 213 can be used to allow clamp 204 to be securely attached to back stem 304 (FIGS. 9 and 10) on grip assembly 300. Preferably, beveled areas 213 are formed on clamp base 202 by conventional forming techniques such as grinding, forming, molding or the like.

With respect to clamp 204, as shown in FIGS. 6, 7 and 10, clamp 204 includes, in part, notches 205 and beveled area 207. As will be discussed in greater detail later, notches 205 and beveled area 207 can be used to allow clamp 204 to be securely attached to back stem 304 (FIGS. 9 and 10) on grip assembly 300. Preferably, notches 205 and beveled area 207 are formed on clamp 204 by conventional forming techniques such as grinding, forming, molding or the like. In another embodiment, an internal surface of the clamp 204 can include a textured surface and/or raised areas 215 that will assist in securely retaining the grip assembly 300.

With respect to fastener 206, as shown in FIGS. 6, 7 and 10, fastener 206 includes, in part, beveled areas 209 and threaded area 211. As will be discussed in greater detail later, beveled areas 209 and threaded area 211 can be used to interact with clamp 204 and threaded area 212 to securely attach back stem 304 to removable, adjustable grip clamp assembly 200.

It is to be understood that a length of push rod 52 may need to be adjusted or the push rod 52 may need to be replaced with a longer push rod so that push rod 52 can properly interact with the tattoo needle cartridge 306 (FIG. 9).

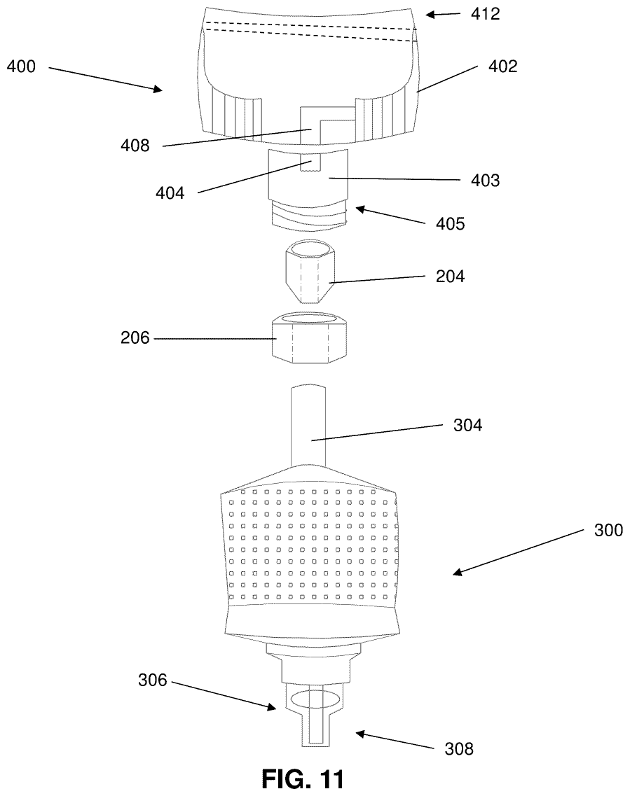

With respect to FIG. 11, there is illustrated another embodiment of a removable, adjustable grip clamp assembly 400. In particular, removable, adjustable grip clamp assembly 400 includes, in part, clamp base 402, clamp 204, and fastener 206. Preferably, removable, adjustable grip clamp assembly 400 is constructed of any suitable, durable, high strength, rust resistant, UV resistant, lightweight material. It is to be understood that clamp 204 and fastener 206 are constructed and are operated in the same manner as discussed above. Also, it is to be understood that removable, adjustable grip clamp assembly 400, grip assembly 300, clamp 204, and fastener 206 are constructed and assembled together to allow needle 308 to extend from motor assembly 50 and out of tattoo needle cartridge 306.

A unique aspect of the present invention is that removable, adjustable grip clamp assembly 400 will allow the pen style tattoo machine to accept and utilize disposable tattoo machine grips that are used normally on traditional style tattoo machines. Not only will this allow the tattoo artists to use the disposable tattoo machine grips with pen style tattoo machines, but it will also does not allow for the disposable tattoo machine grip to spin during the tattoo application process in order to change the distance the tattoo machine needle 308 hangs out of the tip of the tattoo needle cartridge 306 (FIG. 11).

With respect to clamp base 402, as shown in FIG. 11, in one embodiment, clamp base 402 includes, in part, groove 408 and threaded area 412. As discussed in greater detail later, threaded area 412 is located on clamp base 402 to interact with motor assembly connector 102 (FIG. 6) so that removable, adjustable grip clamp assembly 400 can be quickly and easily removed from motor assembly 50 and quickly and securely attached to motor assembly 50. Preferably, threaded area 412 is formed on clamp base 402 by conventional forming techniques such as grinding, forming, molding or the like.

Also, groove 408 is located on clamp base 402 so that groove 408 can interact with notch 404 on clamp 403. As will be discussed in greater detail later, notch 404 is configured to be able to be located within groove 408. Clamp 403 is then rotated in order to secure notch 404 within groove 408. As further shown in FIG. 11, clamp 403 includes threaded area 405. As discussed in greater detail later, threaded area 405 interacts with fastener 206 to allow clamp 204 to securely attach the grip assembly 300 (at back stem 304) to the removable, adjustable grip clamp assembly 400.

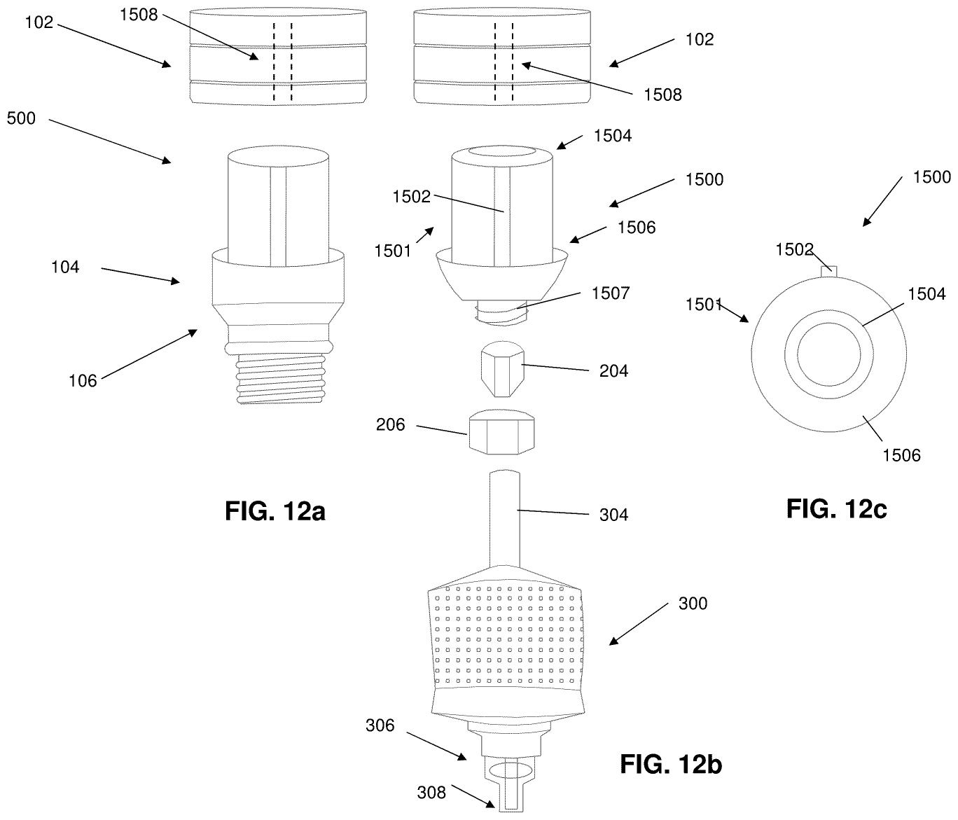

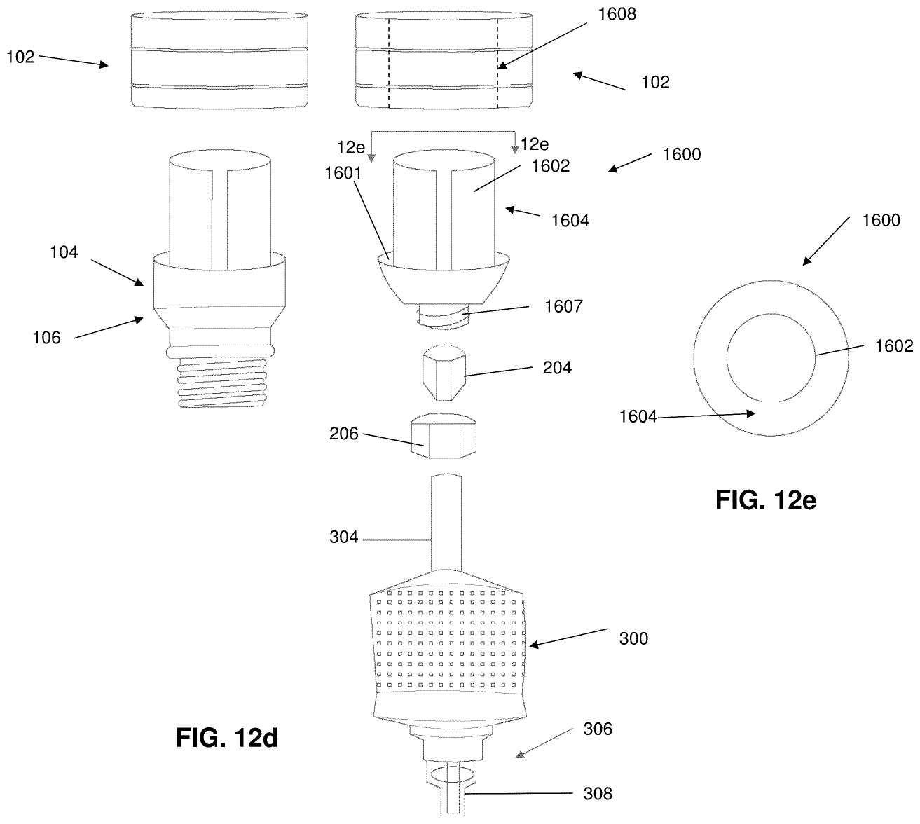

With respect to FIGS. 12a-12c, in one embodiment, there is illustrated another removable, adjustable grip clamp assembly 1500. In particular, removable, adjustable grip clamp assembly 1500 includes, in part, clamp base 1501, clamp 204, and fastener 206. Preferably, removable, adjustable grip clamp assembly 1500 is constructed of any suitable, durable, high strength, rust resistant, UV resistant, lightweight material.

A unique aspect of the present invention is that removable, adjustable grip clamp assembly 1500 will allow any pen style tattoo machine to accept and utilize disposable tattoo machine grips that are used normally on traditional style tattoo machines. This will allow the tattoo artists to use the disposable tattoo machine grips with pen style tattoo machines.

With respect to clamp base 1501, as shown in FIGS. 12b and 12c, in one embodiment, clamp base 1501 includes, in part, notch 1502, magnet 1504, and magnet 1506. As discussed in greater detail later, notch 1502 and magnets 1504 and 1506 are located on clamp base 1501 to interact with motor assembly connector 102 so that removable, adjustable grip clamp assembly 1500 can be quickly and easily removed from motor assembly 50 and quickly and securely attached to motor assembly 50. In particular, the notch 1502 is configured to be located with groove 1508 in motor assembly connector 102 (FIG. 12b). The magnets 1504 and 1506 are then used to securely attach the clamp base 1501 to the motor assembly connector 102. Furthermore, the location of the notch 1502 within the groove 1508 should substantially prevent any unwanted spinning of the grip assembly 300. Preferably, notch 1502 is formed on clamp base 1501 by conventional forming techniques such as grinding, forming, molding or the like.

With respect to clamp 204, as discussed above, clamp 204 is configured to be securely attached to back stem 304 (FIG. 12b) on grip assembly 300. Also, with respect to fastener 206, fastener 206 is configured to interact with clamp 204 and threaded area 1507 to securely attach back stem 304 to removable, adjustable grip clamp assembly 1500.

With respect to FIGS. 12d and 12e, in one embodiment, there is illustrated another removable, adjustable grip clamp assembly 1600. In particular, removable, adjustable grip clamp assembly 1600 includes, in part, clamp base 1601, clamp 204, and fastener 206. Preferably, removable, adjustable grip clamp assembly 1600 is constructed of any suitable, durable, high strength, rust resistant, UV resistant, lightweight material.

A unique aspect of the present invention is that removable, adjustable grip clamp assembly 1600 will allow any pen style tattoo machine to accept and utilize disposable tattoo machine grips 300 that are used normally on traditional style tattoo machines. This will allow the tattoo artists to use the disposable tattoo machine grips with any pen style tattoo machines.

With respect to removable, adjustable grip clamp assembly 1600, as shown in FIGS. 12d and 12e, in one embodiment, removable, adjustable grip clamp assembly 1600 includes, in part, clamp base 1601 and a clamp extension 1602 having a slot or gap 1604 located along a length of clamp extension 1602. As discussed in greater detail later, the extension 1602 is constructed to include a slot or gap 1604. In particular, when the clamp extension 1602 is located within an opening 1608 in the motor assembly connector 102, opening 1608 is sized and shaped so that the extension 1602 will become compressed within the opening 1608 when clamp extension 1602 is located within the opening 1608 in motor assembly connector 102. The compression of the extension 1604 will cause the clamp extension 1602 to be retained within the opening 1608. In this manner, clamp base 1601 will be removably retained within the motor assembly connector 102 so that removable, adjustable grip clamp assembly 1600 can be quickly and easily removed from a motor assembly of a pen style tattoo machine such as motor assembly 50 (FIG. 10) and quickly and securely attached to motor assembly 50. Furthermore, the location of the clamp extension 1602 within the opening 1608 should substantially prevent any unwanted spinning of the grip assembly 300. Preferably, slot or gap 1604 is formed on clamp extension 1602 by conventional forming techniques such as grinding, forming, molding or the like.

With respect to clamp 204, as discussed above, clamp 204 is configured to be securely attached to back stem 304 (FIG. 12d) on grip assembly 300. Also, with respect to fastener 206, fastener 206 is configured to interact with clamp 204 and threaded area 1607 to securely attach back stem 304 to removable, adjustable grip clamp assembly 1600.

### Construction of Removable, Adjustable Grip Clamp Assembly

To construct removable, adjustable grip clamp assembly 200, attention is directed to FIGS. 5-12e. Upon selecting the desired grip assembly 300, the back stem 304 is located adjacent to clamp 204 (FIGS. 9-11 and 12b).

After the back stem 304 is located within clamp 204, clamp 204 is located within the fastener 206 so that fastener 206 will be able to interact with clamp 204 in order to securely retain back stem 304 in clamp 204.

A still another unique aspect of the present invention is the use of clamp 204 to securely retain back stem 304 within clamp 204. In particular, the rotation of fastener 206 on clamp base 202 will cause the threaded areas 211 on fastener 206 to interact with the threaded areas 212 on clamp base 202. Furthermore, the rotation of the fastener 206 on clamp base 202 will cause the beveled areas 209 on fastener 206 to interact with the beveled areas 207 on clamp 204. Since clamp 204 also includes notches 205, the interaction between the beveled areas 209 and 207 will cause the clamp 204 to move inwardly towards the back stem 304. Also, the beveled areas 213 on clamp base 202 will interact with the top of clamp 204 to cause the clamp 204 to also move inwardly towards back stem 304. The fastener 206 is rotated (or tightened) until the fastener 206 cannot be further reasonably rotated (or tightened). In this manner, back stem 304 will be securely retained with clamp 204.

Once the grip assembly 300 has been securely retained within removable, adjustable grip clamp assembly 200, 400, 1500 or 1600, the distance that the tattoo machine needle 308 hangs out of the tip of the tattoo needle cartridge 306 (FIGS. 9, 10 and 12b) can be adjusted, as discussed earlier.

With respect to removable, adjustable grip clamp assembly 400, in order to construct removable, adjustable grip clamp assembly 400, attention is directed to

FIG. 11. Upon selecting the desired grip assembly 300, the back stem 304 is located adjacent to fastener 206.

After the back stem 304 is located within fastener 206, fastener 206 is secured to clamp base 402 through the interaction between clamp 204 and clamp 403 in order to securely retain back stem 304 in clamp base 402. In particular, clamp 204 is located within clamp 403. The fastener 406 is then secured to clamp 403 by threading fastener 206 onto threaded area 405 on clamp 403. This will cause the clamp 204 to contact back stem 304 to cause back stem 304 (and grip assembly 300) to become secured to clamp base 402 in a similar manner as discussed above with respect to FIG. 10.

A still another unique aspect of the present invention is the use of clamp 403. As discussed earlier, once clamp 403 is secured to fastener 206 and clamp 204, clamp 403 is located within clamp base 402 by inserting notch 404 within groove 408 in clamp base 402. The clamp base 402 is then rotated so the notch 404 is securely retained within groove 408. In this manner, grip assembly 300 is securely attached to clamp base 402.

Once the grip assembly 300 has been securely retained within removable, adjustable grip clamp assembly 400, the distance that the tattoo machine needle 308 hangs out of the tip of the tattoo needle cartridge 306 can be adjusted by loosening fastener 206, moving the grip assembly 300 up or down, and then re-tightening fastener 206.

With respect to removable, adjustable grip clamp assembly 1500, in order to construct removable, adjustable grip clamp assembly 1500, attention is directed to FIGS. 12a-12c. As discussed earlier, notch 1502 and magnets 1504 and 1506 are located on clamp base 1501 to interact with motor assembly connector 102 so that removable, adjustable grip clamp assembly 1500 can be quickly and easily removed from motor assembly 50 and quickly and securely attached to motor assembly 50. In particular, the notch 1502 is located with groove 1508 in motor assembly connector 102 (FIG. 12b). The magnets 1504 and 1506 are then used to securely attach the clamp base 1501 to the motor assembly connector 102. Furthermore, the location of the notch 1502 within the groove 1508 should substantially prevent any unwanted spinning of the grip assembly 300. Preferably, notch 1502 is formed on clamp base 1501 by conventional forming techniques such as grinding, forming, molding or the like.

Upon selecting the desired grip assembly 300, the back stem 304 is located adjacent to fastener 206. After the back stem 304 is located within fastener 206, fastener 206 is secured to clamp base 1501 through the interaction between clamp 204 and fastener 206 in order to securely retain back stem 304 in clamp base 1501. In particular, clamp 204 is located within fastener 206. The fastener 206 is then secured to clamp base 1501 by threading fastener 206 onto threaded area 1507 on clamp base 1501. This will cause the clamp 204 to contact back stem 304 to cause back stem 304 (and grip assembly 300) to become secured to clamp base 1501 in a similar manner as discussed above with respect to FIG. 10.

Once the grip assembly 300 has been securely retained within removable, adjustable grip clamp assembly 1500, the distance that the tattoo machine needle 308 hangs out of the tip of the tattoo needle cartridge 306 can be adjusted, as discussed earlier.

### Operation of Removable, Adjustable Grip Clamp Assembly

With respect to the operation of removable, adjustable grip clamp assembly 200, in one embodiment, attention is directed to FIGS. 5-12e. In particular, the tattoo artist selects the desired grip assembly 300 to be attached to the removable, adjustable grip clamp assembly 200.

Upon selecting the desired grip assembly 300, the back stem 304 is located within clamp 204.

After the back stem 304 is located within clamp 204, the fastener 206 is rotated so that fastener 206 interacts with clamp 204 in order to securely retain back stem 304 in clamp 204, as discussed above.

A still another unique aspect of the present invention is the use of clamp 204 to securely retain back stem 304 within clamp 204. In particular, the rotation of fastener 206 on clamp base 202 will cause the threaded areas 211 on fastener 206 to interact with the threaded areas 212 on clamp base 202. Furthermore, the rotation of the fastener 206 on clamp base 202 will cause the beveled areas 209 on fastener 206 to interact with the beveled areas 207 on clamp 204. Since the clamp 204 also includes notches 205, the interaction between the beveled areas 209 and 207 will cause the clamp 204 to move inwardly towards the back stem 304. Also, the beveled areas 213 on clamp base 202 will interact with the top of clamp 204 to also cause the clamp 204 to move inwardly towards back stem 304. The fastener 206 is rotated (or tightened) until the fastener 206 cannot be further reasonably rotated (or tightened). In this manner, back stem 304 will be securely retained with clamp 204.

Once the grip assembly 300 has been securely retained within removable, adjustable grip clamp assembly 200, the distance that the tattoo machine needle 308 hangs out of the tip of the tattoo needle cartridge 306 (FIG. 9) can be adjusted, as discussed earlier.

With respect to removable, adjustable grip clamp assembly 400, In order to operate removable, adjustable grip clamp assembly 400, attention is directed to FIG. 11. Upon selecting the desired grip assembly 300, the back stem extension 304 is located adjacent to fastener 206.

After the back stem 304 is located within fastener 206, fastener 206 is secured to clamp base 402 through the interaction between clamp 204 and clamp 403 in order to securely retain back stem 304 in clamp base 402. In particular, clamp 204 is located within fastener 206 and clamp 403. The fastener 206 is then secured to clamp 403 by threading fastener 206 onto threaded area 405 on clamp 403. This will cause the clamp 204 to contact back stem 304 to cause back stem 304 (and grip assembly 300) to become secured to clamp base 402 in a similar manner as discussed above with respect to FIG. 10.

A still another unique aspect of the present invention is the use of clamp 403. As discussed earlier, once clamp 403 is secured to fastener 206 and clamp 204, clamp 403 is located within clamp base 402 by inserting notch 404 within groove 408 in clamp base 402. The clamp base 402 is then rotated so the notch 404 is securely retained within groove 408. In this manner, grip assembly 300 is securely attached to clamp base 402.

Once the grip assembly 300 has been securely retained within removable, adjustable grip clamp assembly 400, the distance that the tattoo machine needle 308 hangs out of the tip of the tattoo needle cartridge 306 can be adjusted by loosening fastener 206, moving the grip assembly 300 up or down, and then re-tightening fastener 206.

With respect to removable, adjustable grip clamp assembly 1500, in order to operate removable, adjustable grip clamp assembly 1500, attention is directed to FIGS. 12a-12c. As discussed earlier, notch 1502 and magnets 1504 and 1506 are located on clamp base 1501 to interact with motor assembly connector 102 so that removable, adjustable grip clamp assembly 1500 can be quickly and easily removed from motor assembly 50 (FIG. 3) and quickly and securely attached to motor assembly 50. In particular, the notch 1502 is located with groove 1508 in motor assembly connector 102 (FIG. 12b). The magnets 1504 and 1506 are then used to securely attach the clamp base 1501 to the motor assembly connector 102. Furthermore, the location of the notch 1502 within the groove 1508 should substantially prevent any unwanted spinning of the grip assembly 300. Preferably, notch 1502 is formed on clamp base 1501 by conventional forming techniques such as grinding, forming, molding or the like.

Upon selecting the desired grip assembly 300, the back stem 304 is located adjacent to fastener 206. After the back stem 304 is located within fastener 206, fastener 206 is secured to clamp base 1501 through the interaction between clamp 204 and fastener 206 in order to securely retain back stem 304 in clamp base 1501. In particular, clamp 204 is located within fastener 206. The fastener 206 is then secured to clamp base 1501 by threading fastener 206 onto threaded area 1507 on clamp base 1501. This will cause the clamp 204 to contact back stem 304 to cause back stem 304 (and grip assembly 300) to become secured to clamp base 1501 in a similar manner as discussed above with respect to FIG. 10.

Once the grip assembly 300 has been securely retained within removable, adjustable grip clamp assembly 1500, the distance that the tattoo machine needle 308 hangs out of the tip of the tattoo needle cartridge 306 can be adjusted, as discussed earlier.

### Coil Style Tattoo Machine

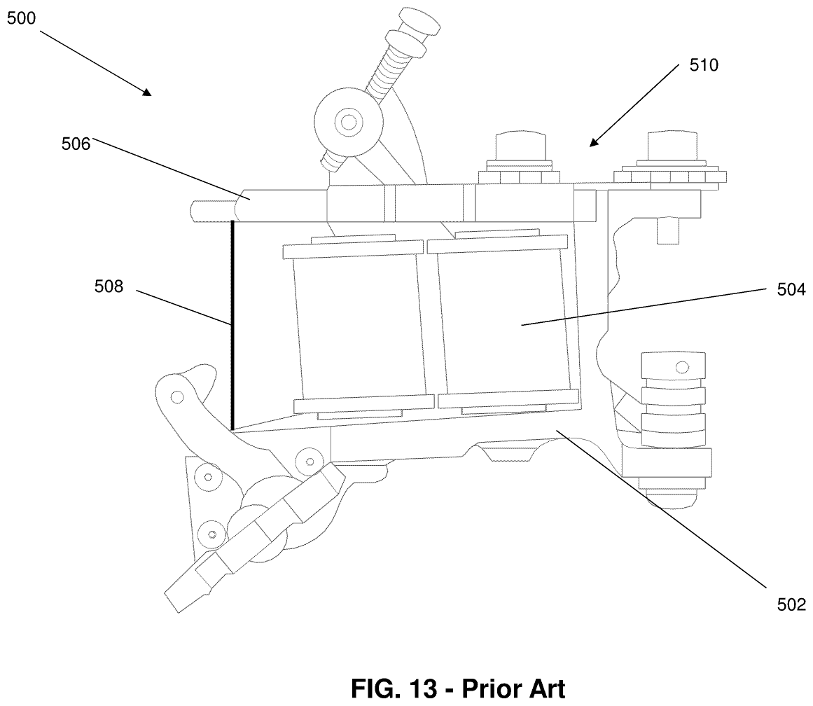

As shown in FIGS. 13, there is illustrated a coil style tattoo machine 500. In particular, coil style tattoo machine 500 includes frame 502, a plurality of coils 504 with each coil 504 surrounding a magnet 510, armature bar 506, and needle 508. It is known that coil style tattoo machine 500 operates by having an alternating electromagnetic current that passes through coils 504 at the top of the machine 500, thereby engaging and disengaging the magnet 510. This engaging/disengaging pulls spring-loaded armature bar 506 and creates an up and down motion, which results in the armature bar 506 tapping the needle 508 into the skin.

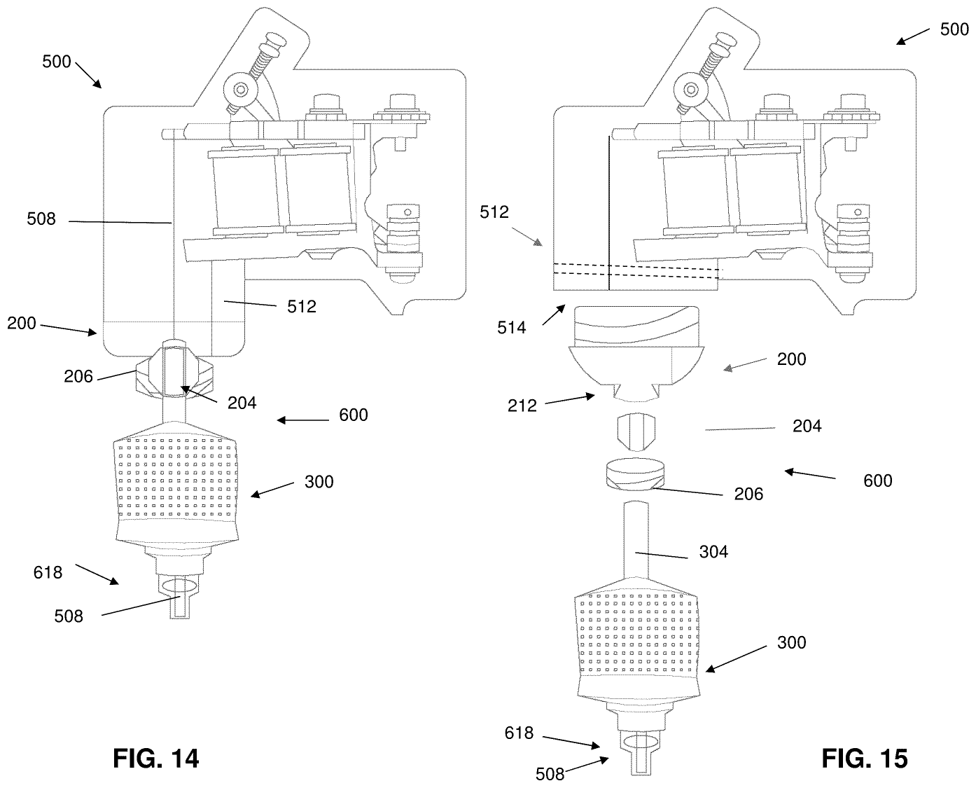

With respect to FIGS. 14 and 15, there is illustrated another embodiment of a removable, adjustable grip clamp assembly 600. As shown in FIGS. 14 and 15, coil style tattoo machine 500 has been modified to include extension 512 having internal threaded area 514. A grip assembly 600 can be attached to coil style tattoo machine 500 through extension 512, removable, adjustable grip clamp assembly 200, clamp 204 and fastener 206. As is well known in tattoo art, the coil style tattoo machine 500 is utilized to move an ink laden tattoo needle 508 out of a tattoo needle cartridge 306 up and down in order to create the desired tattoo graphic. Also, it is to be understood that removable, adjustable grip clamp assembly 200, grip assembly 300, clamp 204, and fastener 206 are constructed and assembled together in order to allow needle 508 to extend from coil style tattoo machine 500 and out of tattoo needle cartridge 306.

It is to be understood that while pen style tattoo machine 2 and coil style tattoo machine 500 are illustrated, a unique aspect of the present invention is that any of the removable, adjustable grip clamp assemblies disclosed in the present invention can be used with either the pen style tattoo machine 2 or coil style tattoo machine 500 in order to attach the grip assembly to tattoo machine. Furthermore, it is to be understood that any tattoo machine that causes a tattoo needle to move up and down in order to create a tattoo can be used with the removable, adjustable grip clamp assemblies and the grip assemblies, as disclosed in the present invention.

With respect to clamp 204 and fastener 206, it is to be understood that clamp 204 and fastener 206 are constructed and are operated in the same manner as discussed above. However, in this embodiment, fastener 206 is releasably connected to removable, adjustable grip clamp assembly 200 at threaded area 212 and removable, adjustable grip clamp assembly 200 is releasably connected to extension 512 through an interaction with threaded area 514 on extension 512 in order to securely retain the coil style tattoo machine 500 to the removable, adjustable grip clamp assembly 600.

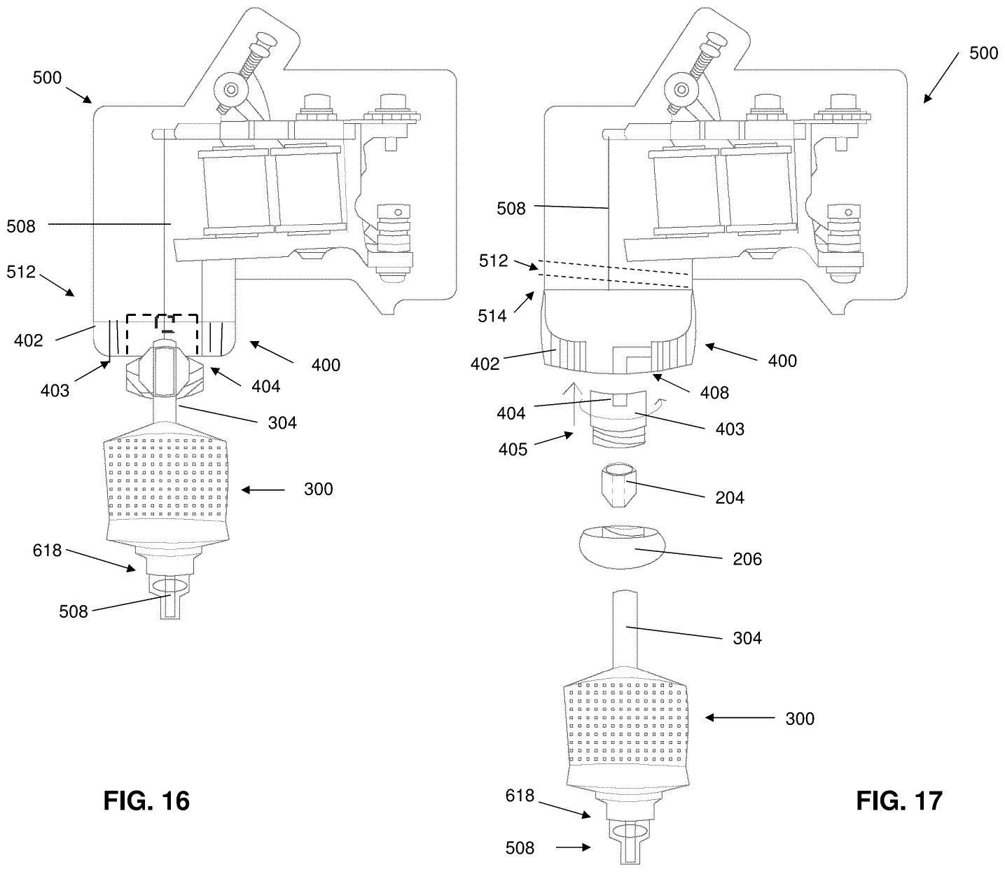

With respect to FIGS. 16 and 17, there is illustrated another embodiment of a removable, adjustable grip clamp assembly 400 being removably attached to coil style tattoo machine 500. As discussed above, removable, adjustable grip clamp assembly 400 includes, in part, clamp base 402, clamp 204, and fastener 206. Preferably, removable, adjustable grip clamp assembly 400 is constructed of any suitable, durable, high strength, rust resistant, UV resistant, lightweight material. It is to be understood that clamp assembly 400, clamp 204 and fastener 206 are constructed and are operated in the same manner as discussed above. Also, it is to be understood that removable, adjustable grip clamp assembly 400, grip assembly 300, clamp 204, and fastener 206 are constructed and assembled together to allow needle 508 to extend from coil style tattoo machine 500 and out of tattoo needle cartridge 306.

A unique aspect of the present invention is that removable, adjustable grip clamp assembly 400 will allow the coil style tattoo machine 500 to accept and utilize disposable tattoo machine grips that are used normally on traditional style tattoo machines. Not only will this allow the tattoo artists to use the disposable tattoo machine grips with coil style tattoo machines, but it will also does not allow for the disposable tattoo machine grip to spin during the tattoo application process in order to change the distance the tattoo machine needle 308 hangs out of the tip of the tattoo needle cartridge 306 (FIGS. 16 and 17).

With respect to clamp base 402, as discussed above, in one embodiment, clamp base 402 includes, in part, groove 408 and threaded area 412. As discussed in greater detail later, threaded area 412 (FIG. 11) is located on clamp base 402 to interact with threaded area 514 on extension 512 on coil style tattoo machine 500 (FIG. 17) so that removable, adjustable grip clamp assembly 400 can be quickly and easily removed from coil style tattoo machine 500 and quickly and securely attached to coil style tattoo machine 500. Preferably, threaded area 412 is formed on clamp base 402 by conventional forming techniques such as grinding, forming, molding or the like.

Also, as discussed above, groove 408 is located on clamp base 402 so that groove 408 can interact with notch 404 on clamp 403. As will be discussed in greater detail later, notch 404 is configured to be able to be located within groove 408. Clamp 403 is then rotated to secure notch 404 within groove 408. As also previously discussed, clamp 403 includes threaded area 405. As discussed in greater detail later, threaded area 405 interacts with fastener 206 to allow clamp 204 to securely attach the grip assembly 300 (at back stem 304) to the removable, adjustable grip clamp assembly 400.

With respect to clamp 204, as discussed above, clamp 204 is configured to be securely attached to back stem 304 (FIG. 16) on grip assembly 300. Also, with respect to fastener 206, fastener 206 is configured to interact with clamp 204, clamp 403, and clamp base 402 to securely attach back stem 304 to removable, adjustable grip clamp assembly 400.

### Construction of Removable, Adjustable Grip Clamp Assembly 600

To construct removable, adjustable grip clamp assembly 600 for use with coil style tattoo machine 500, attention is directed to FIGS. 1417. Upon selecting the desired grip assembly 300, the back stem 304 is located adjacent to fastener 206.

After the back stem 304 is located within fastener 206, fastener 206 interacts with back stem 304, clamp 204, threaded area 212, and extension 512 to securely retain back stem 304 on extension 512 (FIGS. 14 and 15). In particular, clamp 204 is located within fastener 206. The fastener 206 is then secured to threaded area 212 by threading fastener 206 onto threaded area 212. Adjustable grip clamp assembly 200 is then secured to extension 512 by an interaction between adjustable grip clamp assembly 200 and threaded area 514. This will cause the clamp 204 to contact back stem 304 to cause back stem 304 (and grip assembly 300) to become secured to coil style tattoo machine 500 in a similar manner as discussed above with respect to FIG. 10. The fastener 206 is rotated (or tightened) until the fastener 206 cannot be further reasonably rotated (or tightened). In this manner, back stem 304 will be securely retained with clamp 204 and fastener 206.

As shown in FIGS. 16 and 17, as discussed above, in one embodiment, clamp base 402 includes, in part, groove 408 and threaded area 412 (FIG. 11). As discussed in greater detail later, threaded area 412 is located on clamp base 402 to interact with threaded area 514 on extension 512 on coil style tattoo machine 500 (FIG. 17) so that removable, adjustable grip clamp assembly 400 can be quickly and easily removed from coil style tattoo machine 500 and quickly and securely attached to coil style tattoo machine 500. Preferably, threaded area 412 is formed on clamp base 402 by conventional forming techniques such as grinding, forming, molding or the like.

Also, as discussed above, groove 408 is located on clamp base 402 so that groove 408 can interact with notch 404 on clamp 403. As will be discussed in greater detail later, notch 404 is configured to be able to be located within groove 408. Clamp 403 is then rotated to secure notch 404 within groove 408. As also previously discussed, clamp 403 includes threaded area 405. As discussed in greater detail later, threaded area 405 interacts with fastener 206 to allow clamp 204 to securely attach the grip assembly 300 (at back stem 304) to the removable, adjustable grip clamp assembly 400.

With respect to clamp 204, as discussed above, clamp 204 is configured to be securely attached to back stem 304 (FIG. 16) on grip assembly 300. Also, with respect to fastener 206, fastener 206 is configured to interact with clamp 204, clamp 403, and clamp base 402 to securely attach back stem 304 to removable, adjustable grip clamp assembly 400.

Once the grip assembly 300 has been securely retained within removable, adjustable grip clamp assembly 600, the distance that the tattoo machine needle 508 hangs out of the tip of the tattoo needle cartridge 618 can be adjusted, as discussed earlier.

### Operation of Removable, Adjustable Grip Clamp Assembly 600

With respect to the operation of removable, adjustable grip clamp assembly 600, in one embodiment, attention is directed to FIGS. 13-17. In particular, the tattoo artist selects the desired grip assembly 300 to be attached to the removable, adjustable grip clamp assembly 600.

Upon selecting the desired grip assembly 300, the back stem 304 is located within fastener 206.

After the back stem 304 is located within fastener 206, fastener 206 interacts with back stem 304, clamp 204, threaded area 212, and extension 512 in order to securely retain back stem 304 on extension 512 (FIGS. 14 and 15). In particular, clamp 204 is configured to be able to be located within fastener 206. The fastener 406 is then secured to threaded area 212 by threading fastener 206 onto threaded area 212. Adjustable grip clamp assembly 200 is then secured to extension 512 by an interaction between adjustable grip clamp assembly 200 and threaded area 514. This will cause the clamp 204 to contact back stem 304 to cause back stem 304 (and grip assembly 300) to become secured to coil style tattoo machine 500 in a similar manner as discussed above with respect to FIG. 10. The fastener 206 is rotated (or tightened) until the fastener 206 cannot be further reasonably rotated (or tightened). In this manner, back stem 304 will be securely retained with clamp 204 and fastener 206.

As shown in FIGS. 16 and 17, as discussed above, in one embodiment, clamp base 402 includes, in part, groove 408 and threaded area 412 (FIG. 11). As discussed in greater detail later, threaded area 412 is located on clamp base 402 to interact with threaded area 514 on extension 512 on coil style tattoo machine 500 (FIG. 17) so that removable, adjustable grip clamp assembly 400 can be quickly and easily removed from coil style tattoo machine 500 and quickly and securely attached to coil style tattoo machine 500. Preferably, threaded area 412 is formed on clamp base 402 by conventional forming techniques such as grinding, forming, molding or the like.

Also, as discussed above, groove 408 is located on clamp base 402 so that groove 408 can interact with notch 404 on clamp 403. As will be discussed in greater detail later, notch 404 is configured to be able to be located within groove 408. Clamp 403 is then rotated to secure notch 404 within groove 408. As also previously discussed, clamp 403 includes threaded area 405. As discussed in greater detail later, threaded area 405 interacts with fastener 206 to allow clamp 204 to securely attach the grip assembly 300 (at back stem 304) to the removable, adjustable grip clamp assembly 400.

With respect to clamp 204, as discussed above, clamp 204 is configured to be securely attached to back stem 304 (FIG. 16) on grip assembly 300. Also, with respect to fastener 206, fastener 206 is configured to interact with clamp 204, clamp 403, and clamp base 402 to securely attach back stem 304 to removable, adjustable grip clamp assembly 400.

Once the grip assembly 300 has been securely retained within removable, adjustable grip clamp assembly 400 or 600, the distance that the tattoo machine needle 508 hangs out of the tip of the tattoo needle cartridge 618 can be adjusted, as discussed earlier.

While it has not been mentioned, one familiar with the art would realize that the device is not limited by the materials used to create each apparatus that comprises the invention. Any other material type can comprise some or all of the elements in constructing a detachable document holder system in various embodiments of the present invention.

Although the present invention has been illustrated and described herein with reference to preferred embodiments and specific examples, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A pen style tattoo machine having a removable, adjustable grip clamp assembly, comprising:
a motor assembly having a tattoo needle;
a grip assembly having a back stem located at one end of the grip assembly and a tattoo needle cartridge located at another end of the grip assembly, wherein the grip assembly is configured to allow the tattoo needle to extend out of the tattoo needle cartridge; and
a removable, adjustable grip clamp assembly removably attached to the motor assembly and the back stem on the grip assembly.

2. The pen style tattoo machine having a removable, adjustable grip clamp assembly, according to claim 1, wherein the removable, adjustable grip clamp assembly further comprises:
a first clamp base;
a first fastener removably attached to the first clamp base; and
a first clamp located between the first clamp base and the first fastener, wherein the first clamp is configured to contact the back stem.

3. The pen style tattoo machine having a removable, adjustable grip clamp assembly, according to claim 2, wherein the first clamp base further comprises:
a first threaded area located around a periphery on a first end of the first clamp base;
a button spring located adjacent to the first end of the first clamp base;
an O-ring located adjacent to the button spring; and
a first plurality of beveled areas located adjacent to a second end of the first clamp base, wherein the first plurality of beveled areas is configured to interact with one end of the first clamp.

4. The pen style tattoo machine having a removable, adjustable grip clamp assembly, according to claim 3, wherein the first fastener further comprises:
a second plurality of beveled areas located adjacent to one end of the first fastener, wherein the second plurality of beveled areas is configured to interact with another end of the first clamp.

5. The pen style tattoo machine having a removable, adjustable grip clamp assembly, according to claim 1, wherein the removable, adjustable grip clamp assembly further comprises:
a second clamp base;
a second clamp removably attached to the second clamp base;
a second fastener removably attached to the second clamp; and
a third clamp located between the second clamp and the second fastener, wherein the third clamp is configured to contact the back stem.

6. The pen style tattoo machine having a removable, adjustable grip clamp assembly, according to claim 5, wherein the second clamp base further comprises:
a second threaded area located around a periphery on a first end of the second clamp base; and
a first groove located adjacent to a second end of the second clamp base.

7. The pen style tattoo machine having a removable, adjustable grip clamp assembly, according to claim 6, wherein the second clamp further comprises:
a first notch located along one end of the second clamp, wherein the first notch is configured to be retained within the first groove; and
a third threaded area located around a periphery on another end of the second clamp, wherein the third threaded area is configured to interact with the second fastener.

8. The pen style tattoo machine having a removable, adjustable grip clamp assembly, according to claim 1, wherein the removable, adjustable grip clamp assembly further comprises:
a third clamp base having a second notch located along a length of the third clamp base, a first magnet located at one end of the third clamp base, and a second magnet around a periphery of the third clamp base;
a third fastener removably attached to the third clamp base; and
a fourth clamp located between the third clamp base and the third fastener, wherein the fourth clamp is configured to contact the back stem.

9. The pen style tattoo machine having a removable, adjustable grip clamp assembly, according to claim 8, wherein the motor assembly further comprises:
a first drive assembly having a motor assembly connector, wherein the motor assembly connector includes a second groove such that the second notch is configured to be located within the second groove.

10. The pen style tattoo machine having a removable, adjustable grip clamp assembly, according to claim 1, wherein the wherein the removable, adjustable grip clamp assembly further comprises:
a fourth clamp base
a clamp extension operatively connected at one end to the fourth clamp base; and
a slot located along a portion of the clamp extension.

11. The pen style tattoo machine having a removable, adjustable grip clamp assembly, according to claim 10, wherein the motor assembly further comprises:
a second drive assembly having a second motor assembly connector, wherein the second motor assembly connector includes an opening such that the clamp extension is configured to be releasably retained within the opening.

12. A method of constructing a pen style tattoo machine having a removable, adjustable grip clamp assembly, comprising:
providing a motor assembly having a tattoo needle;
providing a grip assembly having a back stem located at one end of the grip assembly and a tattoo needle cartridge located at another end of the grip assembly, wherein the grip assembly is configured to allow the tattoo needle to extend out of the tattoo needle cartridge; and
attaching a removable, adjustable grip clamp assembly to the motor assembly and the back stem on the grip assembly.

13. The method, according to claim 12, wherein the removable, adjustable grip clamp assembly further comprises:
providing a first clamp base;
removably attaching a first fastener to the first clamp base; and
providing a first clamp between the first clamp base and the first fastener, wherein the first clamp is configured to contact the back stem.

14. The method, according to claim 13, wherein providing the first clamp base further comprises:
providing a first threaded area located around a periphery on a first end of the first clamp base;
providing a button spring adjacent to the first end of the first clamp base;
providing an O-ring adjacent to the button spring; and
providing a first plurality of beveled areas adjacent to a second end of the first clamp base, wherein the first plurality of beveled areas is configured to interact with one end of the first clamp.

15. The method, according to claim 14, wherein the first fastener further comprises:
providing a second plurality of beveled areas adjacent to one end of the first fastener, wherein the second plurality of beveled areas is configured to interact with another end of the first clamp.

16. The method, according to claim 12, wherein the removable, adjustable grip clamp assembly further comprises:
providing a second clamp base;
removably attaching a second clamp to the second clamp base;
removably attaching a second fastener to the second clamp; and
providing a third clamp between the second clamp and the second fastener, wherein the third clamp is configured to contact the back stem.

17. The method, according to claim 16, wherein providing the second clamp base further comprises:
providing a second threaded area around a periphery on a first end of the second clamp base; and
providing a first groove adjacent to a second end of the second clamp base.

18. The method, according to claim 17, wherein the second clamp further comprises:
providing a first notch along one end of the second clamp, wherein the first notch is configured to be retained within the first groove; and
providing a third threaded area around a periphery on another end of the second clamp, wherein the third threaded area is configured to interact with the second fastener. 19 The method, according to claim 12, wherein the removable, adjustable grip clamp assembly further comprises:
providing a third clamp base having a second notch located along a length of the third clamp base, a first magnet located at one end of the third clamp base, and a second magnet around a periphery of the third clamp base;
removably attaching a third fastener to the third clamp base; and
providing a fourth clamp between the third clamp base and the third fastener, wherein the fourth clamp is configured to contact the back stem.

20. The method, according to claim 19, wherein the motor assembly further comprises:
providing a first drive assembly having a motor assembly connector, wherein the motor assembly connector includes a second groove such that the second notch is configured to be located within the second groove.
